(19) United States
(12) Patent Application Publication
(10) Pub. No.: US 2002/0183297 A1
(43) Pub. Date: Dec. 5, 2002

(54) Pharmaceutical composition for the treatment of alopecia

(76) Inventor: Sarfaraz K. Niazi, Deerfield, IL (US)

Correspondence Address: GERALD T. SHEKLETON, ESQ., WELSH & KATZ, LTD., 22ND FLOOR, 120 SOUTH RIVERSIDE PLAZA, CHICAGO, IL, 60606, US

(21) Appl. No.: 10/077,289

(22) Filed: Feb. 15, 2002

Related U.S. Application Data
(63) Continuation of application No. 09/681,189, filed on Feb. 14, 2001, now abandoned.

(51) Int. Cl. A61K 31/56

(52) U.S. Cl. 514/178

(57) ABSTRACT

Pharmaceutical compositions containing phystosterols and/or blood flow stimulants are described to promote hair growth through stimulation of follicular cells, bulb cells and stem cells in the scalp to treat the condition of alopecia in humans and animals.

### DETAILED DESCRIPTION

Human hair is the keratin-containing threadlike outgrowth extending from hair follicles in the skin. In humans, hair generally serves protective, sensory, and sexual attractiveness functions. A mature hair shaft is composed of three, and sometimes four, basic structures. The cuticle is the thick outer protective covering consisting of flat overlapping scale-like layers. The cortex is located inside, and is surrounded by, the cuticle. The cortex contains fibrous proteins, which are aligned along the length of the hair axis. Thicker hairs often contain one or more porous regions the medulla located near or at the center of the hair shaft. The fourth basic component is the intercellular cement, which glues or binds the cells together and provides the main pathway for diffusion into the hair fibers. Melanocytes, which produce melanin, the pigment responsible for hair color, are generally contained in the cortex and the base of the bulb of the hair shaft. Essential nutrients and oxygen are carried to the growing hair through capillaries around the base of the bulb. The hair follicle cycle is a complex process and entails involvement of cell differentiation, epithelial-mesenchymal interactions, stem cell augmentation, pattern formation, apoptosis, cell and organ growth cycles, and pigmentation. The most important theme in studying the cycling of hair follicle is that the follicle is a regenerating system. By traversing the phases of the cycle (growth, regression, resting, shedding, then growth again), the follicle demonstrates the unusual ability to completely regenerate itself. The basis for this regeneration rests in the unique follicular epithelial and mesenchymal components and their interactions. Recently, some of the molecular signals making up these interactions have been defined. They involve gene families also found in other regenerating systems such as fibroblast growth factor, transforming growth factor-β Wnt pathway, Sonic hedgehog, neurotrophins, and homeobox. (K S Stenn and R Pauls, Physiol Rev 2001 Jan; 81(1):449-494).

Normal hair follicles cycle between a growth stage (anagen), a degenerative stage (catagen), and a resting stage (telogen). The scalp hairs have a relatively long life cycle: the anagen stage ranges from two to five years, the catagen stage ranges from a few days to a few weeks, and the telogen stage is approximately three months (Fitzpatrick, T. B., et al., eds., DERMATOLOGY IN GENERAL MEDICINE (Vol. I), McGraw-Hill, Inc., 1993, pp. 290-291; Sperling, L. C., J. Amer. Acad. Dermatology (v. 25, No. 1, Part 1), pp. 1-17 (1991)). Shorter hairs found elsewhere on the body have corresponding shorter anagen duration. The morphology of the hair and the hair follicle changes dramatically over the course of the life cycle of the hair. During anagen, the hair follicle is highly active metabolically (Sperling, L. C., J. Amer. Acad. Dermatology (v. 25, No. 1, Part 1), p. 4 (1991)). The follicle comprises a follicular (dermal) papilla at the base of the follicle; epidermal matrix cells surrounding the follicular papilla and forming the base of a hair shaft; and the hair shaft that extends upwards from the papilla through the hair canal (Fitzpatrick, T. B., et al., eds., DERMATOLOGY IN GENERAL MEDICINE (Vol. I), McGraw-Hill, Inc., 1993). The matrix cells are the actively growing portions of the hair (Sperling, L. C., J. Amero Acad. Dermatology (v. 25, No.1, Part 1), p.6 (1991)). At catagen, the matrix cells retract from the papilla, and other degenerative changes occur (Sperling, L. C., J. epithelial cells pushes the keratinized proximal shaft of the hair upwards (Sperling, L. C., J. Amer. Acad. Dermatology (v. 25, No. I, Part I), p. 3 (1999)), and cell death occurs within the follicle (Fitzpatrick, T. B., et al., eds., DERMATOLOGY IN GENERAL MEDICINE (Vol. I), McGraw-Hill, Inc., 1993, p. 291). When the hair follicle reaches the telogen stage, the existing hair has a club-shaped proximal end, and a small bud (a remnant of the epithelial column that is found in catagen) at the base of the follicle (Sperling, L. C., J. Amer. Acad. Dermatology (v. 25, No. I, Part I), p. 3 (1991)). A telogen hair will not grow further (Fitzpatrick, T. B., et al., eds., DERMATOLOGY IN GENERAL MEDICINE (Vol. I), McGraw-Hill, Inc., 1993, p. 291). The pigmentary system that colors hair involves melanocytes located in the matrix area of the follicle, above the follicular papilla (Fitzpatrick, T. B., et al., eds., DERMATOLOGY IN GENERAL MEDICINE (Vol. I), McGraw-Hill, Inc., 1993, p. 292). Melanin pigments produced by the melanocytes flow along dendritic processes (Fitzpatrick, T. B., et al., eds., DERMATOLOGY IN GENERAL MEDICINE (Vol. I), McGraw-Hill, Inc., 1993, p. 292). The dendritic processes are phagocytized by the differentiating matrix cells that become part of the hair shaft; degradation of the phagocytosed material results in release of melanin granules into the cytoplasm (Fitzpatrick, T. B., et al., eds., DERMATOLOGY IN GENERAL MEDICINE (Vol. I), McGraw-Hill, Inc., 1993, p. 671), thus pigmenting the hair. Alterations in normal hair pigmentation or growth may be caused by age, physiologic disease conditions, or injury especially, for example, exposure to ultraviolet-irradiation. The “graying” of hair, both normal (age-associated) and abnormal, is known as canities. Graying results from a progressive decrease in pigment present in the hair shaft, caused by loss of melanocytes (Fitzpatrick, T. B., et al., eds., DERMATOLOGY IN GENERAL MEDICINE (Vol. I), McGraw-Hill, Inc., 1993, p. 671; Gilchrest, B. A., SKIN AND AGING PROCESSES, CRC Press, 1984, p. 19). A decrease in the density of hair follicles is also associated with advancing age (Gilchrest, B. A., SKIN AND AGING PROCESSES, CRC Press, 1984, p. 20). Alopecia areata is a common disease of the hair follicle, affecting about 2% of new patients attending dermatology clinics in the United States and in Britain (Price, V. H., J. Invest. Dermatol., 96:685 (1991)). In alopecia areata, the hair follicle, in response to some unknown signal or injury, is suddenly precipitated into premature telogen, and then cycles in a shortened aborted cycle in which it is repeatedly arrested part way through early anagen. The follicle may remain in this arrested state but is capable of resuming normal growth after months or years. The nature of the signal or injury and the anatomical target for this abnormality are unknown. Histologically, alopecia areata is characterized by peribulbar lymphocytic infiltrate of predominantly T helper cells (Lever, W. F. and Schaumburg- Lever, G., eds., HISTOPATHOLOGY OF THE SKIN, J. B. Lippincott Co., Philadelphia, Pa., 1990, pp. 223-224), strongly suggesting the involvement of the cellular immune system perhaps through a loss of discrimination of self and non-self antigens (Goldsmith, L. A., J.lnvest. Dermatol., 96:985-1005 (1991)). Alternatively, an intrinsic abnormality in the follicular keratinocyte could be activated under the influence of internal or external triggers, which eventually may lead to cellular degeneration and peribulbar inflammatory infiltrate. However, to date no specific antigen has been identified to support the autoimmune theory and no specific intrinsic difference has been reported between normal bulbar and alopecia areata keratinocytes. The hair follicle is an epidermal derivative that undergoes cycles of growth, involution, and rest. The hair cycle has well-orchestrated kinetics regulated by interactions between mesenchymal and epithelial cells, although the intracellular signals remain unclear. There is suggestion that telogen-to-anagen progression required organized keratinocyte migration in response to mesenchymal stimuli.

Alopecia (baldness) a deficiency of hair, either normal or abnormal, is primarily a cosmetic problem in humans. Hair loss occurs in a variety of situations. These situations include male pattern alopecia, alopecia senilis, alopecia areata, diseases accompanied by basic skin lesions or tumors, and systematic disorders such as nutritional disorder and internal secretion disorders. The mechanisms causing hair loss are very complicated, but in some instances can be attributed to aging, genetic disposition, the activation of male hormones, the loss of blood supply to hair follicles, hair that is readily seen. However, in the so-called bald person although there is a noticeable absence of terminal hair the skin does contain vellus hair, which is a fine colorless hair, which may require microscopic examination to determine its presence. This vellus hair is a precursor to terminal hair. In both women and men, the occurrence of an increased loss of hair is accompanied by the fear of becoming totally bald- headed. Besides the medical aspect, disturbances in the hair growth thus present a great personal problem for the affected person. The rate of growth of the hair amounts to about 0.35 mm per day, the hair density is from about 80,000 to 150,000 hairs per head. A loss of 100 hairs per day constitutes already a pathological effluvium. From hair follicles that remained intact, hair is able to re-grow. However, during a multiphase, lengthy re-growth, hair follicles may shrink and lead to a gradual loss of hair.

The existence of a number of pathologic syndromes depends on androgen hormones. An unexplained switch causes androgenic alopecia from the growth promoting effect of androgens on the hair follicles to hair loss. In skin, androgen mediated disorders, such as alopecia, acne vulgaris, and hirsutism, excess of the cutaneous androgens are a major nosological factor. The androgenic hormones can act only via an androgenic receptor, which is a transcription factor, a protein that interacts with a specific region of DNA. Thus, the mode of action of testosterone and its much more potent analog, 5-alpha dihydrotestoterone depends upon binding to the androgenic receptors. Only then can transcription by RNA polymerase II take place. In the treatment of androgenic alopecia, various antiandrogens originally developed for the treatment of prostate cancer were claimed for systemic use, but side effects of chronic therapy with these systemically absorbable substances were of concern. The U.S. Pat. No. 6,184,249 to Sovak, et al., is for the use of substituted phenylalanines that bind specifically to androgen receptor reducing the incidence of alopecia. The U.S. Pat. No. 6,174,892 to Gormley, et al., is for a method of treating and/or reversing androgenic alopecia and promoting hair growth, and methods of treating acne vulgaris, seborrhea, and female hirsutism, by administering to a patient in need of such treatment a 5-a-reductase 2 inhibitor, such as finasteride.

One form of hair loss, alopecia areata, is known to be associated with autoimmune activities; hence, topically administered immunomodulatory compounds demonstrate efficacy for treating that type of hair loss. The immunosuppressant drugs FK506, rapamycin and cyc10sporin ar well known as potent T-cell specific immunosuppressants, and are effective against graft rejection after organ transplantation. Topical application ofFK506 (Yamamoto et al., J. Invest. Dermatol., 1994, 102, 160-164; Jiang et al., J. Invest. Dermatol. 1995, 104,523-525) and cyclosporin (Iwabuchi et al., J. Dermatol. Sci. 1995,9, 64-69) stimulates hair growth in a dose-dependent manner. The hair growth and revitalization effects ofFK506 and related agents are disclosed in many U.S. patents (Goulet et al., U.S. Pat. No. 5,258,389; Luly et al., U.S. Pat. No. 5,457,111; Goulet et al., U.S. Pat. No. 5,532,248; Goulet et al., U.S. Pat. No. 5,189,042; and Ok et aI., U.S. Pat. No. 5,208,241; Rupprecht et al., U.S. Pat. No. 5,284,840; Organ et al., U.S. Pat. No. 5,284,877). Other U.S. patents disclose the use of cyclosporin and related compounds for hair revitalization (Hauer et al., U.S. Pat. No. 5,342,625; Eberle, U.S. Pat. No. 5,284,826; Hewitt et al., U.S. Pat. No. 4,996,193). These patents also relate to compounds useful for treating autoimmune diseases and cite the known use of cyclosporin and related immunosuppressive compounds for hair growth. Honbo et al., in EP 0 423714 A2 disclose the use of relatively large tricyclic compounds, known for their immunosuppressive effects, as hair revitalizing agents. T 0 overcome the side effects of immunosuppressants, several developments have been made using nonimmunosuppressant techniques. Hamilton and Steiner disclose in U.S. Pat. No. 5,614,547 a novel pyrrolidine carboxylate compounds, which bind to the immunophilin FKBPI2 and stimulate nerve growth, but which lack immunosuppressive effects. The U.S. Pat. No. 6,177,455 to Steiner, et al., is for pharmaceutical compositions and methods for treating alopecia and promoting hair growth using non-immunosuppressant pyrrolidine derivatives.

Stem cells are by definition present in all self-renewing tissues. These cells are believed to be long-lived, have it great potential for cell division and are ultimately responsible for the homeostasis of steady-state tissues. Stem cells are normally slow cycling. They can, however, be induced to enter the proliferative pool in response to certain growth stimuli. When stem cells undergo occasional cell division, they give rise to more rapidly proliferating “transient amplifying cells” (“T A”). Stem cells possess many of the following properties: they are relatively undifferentiated, ultrastructurally and biochemically; they have a large proliferative potential and are responsible for the long term maintenance and regeneration of the tissue; they are normally “slow-cycling”, presumably to conserve their proliferative potential and to minimize DNA errors that could occur during replication; they can be stimulated to proliferate in response to wounding and to certain growth stimuli; they are often located in close proximity to a population of rapidly proliferating cells corresponding to the transient amplifying cells (“TA”) in the scheme of (I) stem cell to (2) TA cell to (3) terminally differentiated cell, and they are usually found in well protected, highly vascularized and innervated areas. Positive identification of stem cells has been difficult because, there are few known immunological or biochemical markers specific for epithelial stem cells. Since they are normally “slow-cycling”, they cannot be labeled by single pulse administration of radioactive materials typically used to detect actively proliferating TA cells. The U.S. Pat. No. 5,756,094 to Lavker, et al., describes a method for identification of these cells by labeling these cells continuously to generate label- retaining cells (LRCs). Cotsarelis et al., J. Invest. Dermol. 1 989a, 92(3) disclose a method to facilitate detection of LRCs based on the ability of slow-cycling cells to be recruited to proliferate in response to hyperplastic stimuli.

Stem cells of various epithelia share a common set of features. It is shown that in hair follicles, the heavily pigmented stem cells are located at the base, in close proximity with follicular papillae and associated vasculature. Cotsarelis, et al., Cell 1990,61: 1329-37, show that the hair follicle stem cells were found to exist exclusively in the mid-portion of the follicle at the arrector pili muscle attachment site termed the “bulge” area of the hair follicle.

The demonstration that all the slow-cycling epithelial cells of mouse vibrissa and pelage follicles are concentrated in the bulge area supports the view that follicular epithelial stem cells reside in the upper follicle in the vicinity of the bulge (Cotsarelis et al. 1990 supra; Kobayashi, et al., PNAS USA 1993 90: 7391-5; Rochat, et al., Cell 1994,76: 1063-73; Yang, et al. J. Invest. Derm. 1993, 101: 652-9). Follicular papilla cells have been shown to play an important role in “activating” the normally slow- cycling follicular epithelial stem cells to proliferate resulting in the initiation of anagen (the growing phase of the hair cycle; Cotsarelis, et al., 1990 supra). The molecular mechanism by which the follicular papilla cells actually signal the epithelial stem cells to divide is, however, obscure. Dermal papilla specific messenger RNAs have been identified which encode growth modulating molecules which are synthesized in large quantities by follicular cells (but not by other neighboring cells) and undergo hair-cycle-dependent changes. For example, it was shown in U.S. Pat. No. 5,756,094 that nexin I is a major component of the papillae of growing, but not resting, hair follicles and is important in follicular regulation and hair growth. Nexin I is a potent protease inhibitor that can inactivate a number of growth-regulating serine proteases including thrombin, tissue plasminogen activator and urokinase The bulge cells possess many stem cell properties. They mark the end of the permanent portion of the hair follicle. They possess a relatively primitive cytoplasm. They are normally slow cycling, but can be stimulated to proliferate by tumor promoter, TP A. Finally, they are located in a physically well-protected and well-nourished area. The population of putative stem cells located exclusively in the vicinity of the bulge area is consistent with their being the long-hypothesized pluripotent stem cells, giving rise not only to the hair follicle, but also the sebaceous gland and epidermis. The bulge is a subpopulation of outer root sheath cells located in the mid-portion of the follicle at the arrector pili muscle attachment site. The hair follicle stem cells reside in the matrix or lower bulb area of the hair bulb. The discovery that hair follicle stem cells are involved in skin carcinogenesis has led to the development of methods for identifying and modulating the activity of slow-cycling cells for diagnostic and therapeutic purposes and for populations.

A number of growth factors have been reported to be useful for modulating stem cell activity. For example, cytokines such as Tumor Necrosis Factor (TNF), Epidermal Growth Factor (EGF), Transforming Growth Factor (TGF) and Interleukin-1 (IL-1) are believed to be useful. Cellular targets in acute graft versus host disease have been postulated to be keratinocytes with stem cell properties. Because stem cells are normally slow cycling but proliferate rapidly upon inductive stimulation, they may be attractive targets for cytokines such as TNF. EGF has been shown to have broad biological effects. Most significantly, it has the ability to induce the proliferation of basal keratinocytes. Furthermore, it has been shown to support growth during fetal development and accelerate re-epithelialization during wound healing. TGF-a has been shown to be involved in the regulation of both growth and differentiation of epithelial cells. It is known to stimulate keratinocyte growth in vitro. IL-1 is known to induce proliferative activity in epidermal cells. Keratinocytes of the basal layer of the epidermis express the high affmity (trk E and trk) and the low affmity (P75) NGF receptors (NGF-R). NGF, produced by keratinocytes, protects cells from death when it binds to NGF receptors. In cells, this NGF effect is mediated in part by induction of the protective protein Bcl-2. Interestingly, basal epidermal keratinocytes express Bcl-2 protein. Normal anagen hair follicles strongly express the p75 NGF-R and that p75 NGF-R expression is significantly reduced and limited to a few basal keratinocytes in telogen hair follicles. The U.S. Pat. No. 6,103,689 to Gilchrest, et al., is for a method for maintaining hair growth and coloration in humans by using neurotrophin ligands to prevent p75 nerve growth factor (NGF) receptor mediated apoptosis in melanocytes and keratinocytes.

Messenger RNAs have now been identified which encode growth-modulating molecules which are synthesized by follicular cells (but not by other neighboring cells) and which undergo hair-cycle-dependent concentration changes in the hair follicle. Osteopontin message was also found in cultured follicular dermal papilla cells, but not in cultured fibroblasts. Osteopontin is known to be a major bone matrix protein; however, its presence in follicles was not previously known. Osteopontin is also a secreted protein, which may be involved in the regulation of follicular epithelial growth and hair growth.

Several novel techniques and preparations have been described to promote hair growth based on the various theories and techniques described above. The U.S. Pat. No. 5,607,693 to Bonte, et aI., is for a cosmetic or pharmaceutical composition which comprises oxyacanthine or an extract of a plant in which it is present, such as Berberis vulgaris or barberry. One particular association is that of oxyacanthine with a saponin. This composition can be intended in particular for stimulating hair growth, retarding hair loss or combating pruritus. The U.S. Pat. No. 6,159,475 to Olguin for a hair growth formulation. The two basic main ingredients are castor oil and a special lemon extract. The U.S. Pat. No. 6,149,933 to Nelson is for a dietary supplement, which is useful for the promotion of healthy hair, and pigment restoration in human subjects is provided. The dietary supplement contains a copper salt, p-aminobenzoic acid, pantothenic acid and vitamin B6. The U.S. Pat. No. 6,013,279 to Klett-Loch is for a combination preparation for stimulating the growth of hair and skin and nails with a combination of vitamins, enzymes, and amino acids. To increase the effectiveness of the combination preparation, its use is described as a supplement to a topically applicable hair growth stimulant, in particular a thymus-containing therapeutic agent. The U.S. Pat. No. 5,972,345 to Chizick, et al., is for a natural formulation for treatment of male pattern hair loss. The formulation contains a combination of Saw Palmetto extract, African Pygeum extract, stinging nettle extract, and optionally zinc, vitamin B6 and green tea extract.

In the present invention, compositions are provided which stimulate stem cells and/or bulge cells to create new hair follicular cells, to enhance blood flow to hair follicles resulting in the activation and transition of stem cells to active cells yielding terminal hair growth. The active molecule reported in this invention are naturally occurring phytosterol, particularly, 13 -sitosterol. In general, for topical administration, formulated in combination with one or more excipients. The amount of the growth stimulation molecule needed in order to stimulate stem cell growth varies depending upon the particular individual. Further, the number of applications and the period of time over which the applications are made can vary considerably depending upon the actual state of the follicular cells. However, those skilled in the art can routinely determine the precise amounts, numbers and periods of administration. As a guideline, a composition comprised of less than 1% to greater than 99% weight percent of a growth-stimulating component β-sitosterol is applied topically on a daily basis over a period of several weeks to months in a pharmaceutical dosage form.

β-sitosterol (C 29R 500, molecular weight 414.72) is a common sterol in plants. It is generally isolated from wheat germ or corn oil. Sterols are important cyclized triterpenoids that perform many critical functions in cells. Phytosterols such as campesterol, stigmasterol and β-sitosterol in plants, ergosterol in fungi and cholesterol in animals are each primary components of the cellular and sub-cellular membranes in their respective cell types. The dietary source of phytosterols in humans comes from vegetables and plant oils. The estimated daily phytosterol content in the conventional western-type diet is approximately 250 milligrams in contrast to a vegetable diet, which would provide double that amount. Although having no nutritional value to humans, phytosterols have recently received a great deal of attention due to their possible anti-cancer properties and their ability to decrease cholesterol levels when fed to a number of mammalian species, including humans. Phytosterols aid in limiting cholesterol absorption, enhance biliary cholesterol excretion and shift cholesterol from atherosclerotic plaque. While many of the mechanisms of action remain unknown, the relationship between cholesterol and phytosterols is apparent. This is perhaps not surprising given that chemically, phytosterols closely resemble cholesterol in structure. The major phytosterols are -sitosterol, campesterol and stigmasterol. Others include stigmastanol (β-sitostanol), sitostanol, desmosterol, chalinasterol, poriferasterol, clionasterol and brassicasterol. (Gould R. G., Jones R. J., LeRoyu G. V., Wissler R. W., Taylor C. B.; Absorbability of B-sitosterol in humans; Metabolism, (August) 1969; 18(8): 652-662. Tabata T., Tanaka M., Lio T.; Hypocholesterolemic activity of phytosterol. II; Yakugaku Zasshi, 1980; 100(5): 546-552. Hepistall R. R., Porter K. A.; The effect of β-sitosterol on cholesterol-induced atheroma in rabbits with high blood pressure; Br. J. Experimental Pathology, 1957; 38: 49-54.). The role of phytosterols, particularly, β-sitosterol in stimulating human stem cells and particularly promoting hair growth has not been reported yet.

Several novel applications of p phytosterols including β-sitosterol have been reported. The U.S. Pat. No. 5,965,449 to Novak describes a method of assessing risk for cardiovascular disease and other disorders and phytosterol-based compositions useful in preventing and treating cardiovascular disease and other disorders. The level of serum campesterol and β-sitosterol are determined and their ratio is correlated with the risk of cardiovascular or a related disorder. The U.S. Pat. No. 5,523,087 to Shlyankevich is for a pharmaceutical composition for the treatment of diabetic male sexual dysfunction; it contains physosterogens, phosphatidyl choline, β-sitosterol, Darniana leaf extract and vitamins and minerals. The U.S. Pat. No. 5,486,510 to Bouic, et al., is for a mixture of β-sitosterol glucoside and β-sitosterol is administered to persons for the modulation or control of immune respouses. The U.S. Pat. No. 5,747,464 to See is for a composition for inhibiting absorption of fat and cholesterol from the gut and a method for making and using the composition. The composition comprises β-sitosterol, bound irreversibly to pectin to form a β-sitosterol and pectin complex. The U.S. Pat. No. 5,118,671 to Bombardelli, et al., is for complexes formed between aescin, cholesterol or β-sitosterol and phospholipids and a method for producing an anti-inflammatory effect is also described.

Hair follicular growth is dependent on many factors, one of that is the nutrition provided to follicles. In providing such nutrition, the blood flow to scalp plays an important role. In addition, the absorption of the active ingredient across the follicle is also enhanced if the blood flow to topical tissue is enhanced. The conversion of stem cells to active cells depends also on blood flow to tissue. Thus the treatment of two major classes of components that can accomplish this, a rubefacient compound, chemicals that enhance blood flow to surface by creating an irritation to surface and molecules that by their pharmacological response, either local or systemic, enhance and/or maintain blood flow to specific tissue. The ingredient used in this invention to enhance blood flow to scalp and hair follicles is capsaicin, which has been reported (U.S. Pat. No. 5,384,123) to rejuvenate skin and to act as an aphrodisiac (U.S. Pat. No. 6,039,951). Capsaicin, the active component in hot chili pepper, is known from “Drugs & Aging”, 1995,7 (4), pp 317-328, which discloses a topical composition containing capsaicin with analgesic effect. Whereas the choice of capsaicin is made in this composition as a preferred embodiment, other rubefacient agents such as menthol, mustard (U.S. Pat. No. 5,476,492), nicotinic acid and its various derivatives, methyl salicylate, and a variety of other compounds that enhance blood flow to peripheral surface upon direct application may be used instead. Besides rubefacients, other pharmacological agents known to dilate blood vessels can also be used. The vasodilators used in accordance with the method of the invention may produce vasodilation by any of a wide range of mechanisms. One suitable class of vasodilators is the adrenergic neuron blockers, which interfere with transmission in the nerve. Several nerve types may be acted upon to produce vasodilation depending on the pharmacological category of the agent. The vasodilators in this class include debrisoquine. Further classes of vasodilators act on phannacological receptors on the smooth muscle membrane. These include presynaptic receptor blockers and vasodilators, which reduce the amount of chemical messenger in the synaptic vesicles, which provide the point of contact with the smooth muscle. An example of the former type is clonidine and an example of the latter type is guanethidine. One specific class of vasodilators act on catecholamine transmitters and are termed alpha-adrenergic blocking agents. Example of this type of vasodilator include prazosin, lebetaiol, doxazocin, phenoxybenzaInine, phentolamine, betahistine, ergotamine and sumatriptin. There are several other receptor types present on the smooth muscle cell which mediate contractions and vasodilation results when actuation of these receptors is interfered with renin receptors and angiotensin II receptors mediate such contractions, and agents which block these processes indirectly or directly are Vasodilators. ACE inhibitors and Angiotensin n receptor antagonists include include ibesartan. The ACE Inhibitors include quinapril, captopril, enalapril, perindopril, trandolapril, cilazapril, fosinopril, lisinopril, and rarnipril. There are other nerve processes which mediate contraction—these are the purinergic and neuropeptide Y transmitter and receptor systems and vasodilators which act on these nerve processes may be used in accordance with the invention. Similarly there is a range of receptor types, which may be targeted to provide the vasodilator effect. These include α-adrenergic, α-2-adrenergic, neuropeptide Y and purinergic. A further major class of vasodilators is those, which act directly in the smooth muscle membrane. They include hydrallazine, veraparnil, diltiazem, felodipine, minoxidil, amlodipine, glyceryl trinitrate, isosorbide mononitrate, nicorandil, dipyridamole, multiple actives, alprostadil, oxpentifylline, hydroxyethyl rutosides and tartrazine, adenosine and nimodipine.

Since the purpose of this composition is to achieve substantial penetration off}—sitosterol and/or and appropriate blood flow enhancer across the scalp and hair follicles, the composition includes optionally, an absorption promoter which may include a substantially water-insoluble transdermal penetration enhancing compound selected from the group consisting of C4 to C16 aliphatic group substituted acetals, hemi-acetals and morpholines and further comprising a physiologically acceptable water soluble polar compound selected from the group consisting of alcohols, glycols, lactams, urea, cycloethylene urea, 1,3-dioxolone, 2-methyl-1-3-dioxolone, 1,3-dioxane, 2methyl-1,3-dioxane, morpholine, N-methylmorpholine, N-dimethylfonnamide, dimethylsulfoxide, methylacetate, ethyllactate, monosaccharides, polysaccharides, amino acids, amino alcohols, diethylamine and cycloethylene carbonate. The polar compound may be selected from a group consisting of alcohol, glycol, dioxolane, formamide, carbonate, glucose, urea and mixtures thereof. Alternatively, the polar compound may be an alcohol glycol mixture or lactim. Other compounds include 1-odecylazacycloheptan-2-one hexamethyiene-lauramide, N-methyl-2-pynolidone, a weight of the preparation.

For topical administration, it is preferred that the growth-stimulating composition be formulated in an alcoholic or hydro-alcoholic solution that in itself acts to dissolve or remove sebaceous secretions, which may interfere in the absorption of the active ingredients. The type of formulation and amount of the formulation applied will be determined to a large extent by the caregiver. While a single application of the growth stimulating molecule may be effective, in order to obtain the best results it may be necessary to apply it periodically, such as every day, or every other day depending upon the individual and the state of the cells being treated. Again the amount of the growth stimulating molecule and the frequency at which it is applied, is a matter which can readily be determined by one skilled in the art based upon visual changes observed in hair growth. The method of applying the subject composition can also involve combining the composition with vitamin A, series of vitamin Bs, vitamin C, cyanocobalamin, vitamin E, methionine, cystine or other amino acids, albumin, lactalbumin, selenium or other trace metals, thymus, melatonin, and yeast.

Further the subject composition may be combined with other drugs or food supplements that work to promote conversion or growth of stem cells, enhance blood flow and stimulate hair follicular growth.

The present invention relates to the field of stem cell, bulge cell or hair follicle stimulation and in particular to the field of hair growth stimulation. The hair growth formula has been described with reference to particular embodiments. Other modifications and enhancements can be made without departing from the spirit and scope of the claims that follow.

| | |
|---|---|
| [Example 1] | |
| Ingredient | Concentration β-sitosterol 10% |
| Capsaicin | 0.075% |
| Dimethylsulfoxide | 5% |
| Alcohol USP | qs to 100% |
| [t2] | |
| [Example 2] | |
| Ingredient | Concentration β-sitosterol 10% |
| Methyl nicotinate | 0.3% |
| Dimethylsufloxide | 5% |
| Alcohol USP | qs to 100% |
| [t3] | |
| [Example 3] | |
| Ingredient | Composition |
| Methyl nicotinate | 0.30% |
| Dimethylsulfoxide | 5% |
| Alcohol USP | qs to 100% |
| [t4] | |
| [Example 4] | |
| Ingredient | Concetration |
| Capsaicin | 0.075% |
| Dimethylsufoxide | 5% |
| Alcohol USP | qs to 100% |
| [t5] | |
| [Example 5] 9 | |

The composition is applied to scalp ideally after thoroughly cleansing hair with soap and water to remove as much sebaceous secretions as possible. Sufficient quantity of the composition is applied to balding areas of scalp repeatedly, 1-2 times per day and applications continued for several days or weeks. It may be necessary, as it has been observed, to repeat the application frequently to keep the new follicular growth and to initiate further growth.

The effectiveness of topical vasodilators or drugs that enhance blood flow to scalp tissues can be measured by many techniques. In this study we used the technique of monitoring blood flow and skin temperature using laser Doppler imaging as it has been successfully used to measure skeletal muscle blood flow at rest and during exercise in human subjects (Radergran, G., Proc. Nutr. Soc., 58(4): 887-98, 1999) and to assess microcirculation (Eun, H. C., 13(4): 337-47, 1995). In this invention we studied the effectiveness of capsaicin and methyl nicotinate in improving blood flow to follicular zone.

The effectiveness of stimulant of stem cells can be readily evaluated using the technique of tritiated thymidine labeling. We studied the effectiveness of the invention on stimulation of stem cell populations by using the technique of tritiated thymidine labeling of stem cells. The effects of compositions described here were studied on explants of murine skin. Explant cultures were serially harvested at daily intervals for the first 4 days of exposure, and composition effects on ³H-TdR (tritiated thymidine) incorporation assessed in accordance with standard techniques. Because of the slow- cycling nature of stem cells, repeated administration of tritiated thymidine is necessary. After the labeling, the cells are chased for four weeks wherein the stem cells retain the label longer and are thus quantitated comparatively to control.

In another series of experiments, a cohort of mice was continuously labeled for 2 weeks with ³H- TdR and then allowed to rest for 4 weeks. Once labeled, cells that cycle slowly retained isotopes for an extended period of time. Twice daily, subcutaneous injections of ³H-TdR were given to newborn mice over the first seven days of life resulting in the labeling of almost 100% of nuclei in mouse epidermis, hair follicles, sebaceous glands, fibroblasts, and endothelial cells. Once labeled, cells, which cycle slowly (stem cells) retain the isotope for an extended period of time and are, thus, identified as label retaining cells. Test preparations were applied dermally to labeled animals. Four hours prior to sacrifice, colcemide (4 mg/kg) was injected intraperitoneally. Animals were sacrificed at 2,6, 12 and 24 hours after the application of composition and skin from injected areas fixed and processed for autoradiography according to routine procedures. Appearance of labeled mitotic figures indicated that slow cycling cells (stem cells) have been induced to proliferate.

The direct evidence of effectiveness of the products was further demonstrated using an animal model. The test is based on a study of the activity of the invention on the pilary cycle of Sprague Dawley rats, all of which are 23 days old. The pilary cycles of all the animals are still synchronous at this age. The aim of the test was more particularly to demonstrate the action of the invention on the prolongation of the hair growth phase or so-called “anagenic phase.” This is done in the following manner. On day 24, all the rats are shaved on the sides of the lower part of the back so as to leave only a short length of hair, which is just enough to allow subsequent depilation. From day 25 (age of the rats) to day 65, the test products are then applied daily at a dose, which changes with the weight of the animals. This dose is 0.5 ml on day 25 and reaches 2 ml on day 65. At substantially regular intervals of time (about every 3rd day), starting from day 28, a tuft of hairs is removed from the animal's left side using tweezers. The roots of 10 hairs selected at random from this tuft are observed under high magnification and the number of hairs in the anagenic phase, recognizable by the characteristic shape of the root, is counted. The percentage of hairs in the anagenic phase (growth phase) is thus determined as a function of time on groups of 10 animals. The study was performed on 30 rats divided into 3 groups of 10 animals. The first group receives a preparation according to the invention; the second group received only the excipients. The third group is the control group, which does not receive any product. In all instances the anagenic phase was more prolonged in the rats treated with control group. This was particularly marked from day 37 onwards. Thus it was clear that, by extending the duration of the anagenic phase, the invention described here substantially retards hair loss and promotes renewed growth.

Finally, the compositions were tested in humans. Ten subjects with advanced male pattern baldness used test preparation #1 and #2 (five subjects each) as described above for a period of three weeks. In all instances, while the preparation was applied twice a day, significant growth of additional terminal hair was recorded. Surprisingly, upon cessation of treatment, the hair growth remained and continued, unlike what has been reported in literature on the use of hair growth stimulants whereby cessation of treatment results in loss of new growth. Three female subjects with thinning hair used composition #3 and #4 for a period of five weeks daily. They reported significant increased in hair density, particularly the soft-peach effect. Six male subjects with different degrees of hair loss used composition #5 and #9; significantly higher effect were noted in the use of composition #5 but in both instances, significant peach effect and in the case of composition #5, terminal hair growth was recorded within three weeks of treatment.

The four testing procedures described above established the evidence that compositions containing β-sitosterol significantly enhance the activity of stem cells and/or bulge cells responsible for growth of hair; that compositions containing ingredients known to enhance blood flow when applied to scalp produce an increase in follicular growth and perhaps a stimulation of bulb cells but do not show any significant effect on the proliferation of stem cells. A combination of β-sitosterol and ingredients known to improve blood flow most significantly increases the number of new hair follicles. Compositions containing dimethylsulfoxide as an agent responsible for enhancing penetration of ingredients did not show any significant effect on the activity of ingredients known to enhance blood flow but it had significant effect on the activity of β-sitosterol in stimulating stem cells.

It was established from these studies that β-sitosterol significantly enhances stem cell activity leading to enhance growth of hair in animals and humans. A combination of β-sitosterol with ingredients known to enhance blood flow to the site of application and the ingredients known to enhance penetration of drugs across biological membranes further enhances the utility of β-sitosterol. It was also observed that compositions containing known ingredients that enhance blood flow, when applied directly to scalp, enhances growth of hair. The exact dose and mode of application can vary among individuals and anyone with requisite knowledge about treatment of human ailments should be able to judge and thus recommend an appropriate dosing of these compositions.

2. A composition for stimulating the growth of hair by enhancing blood flow to scalp, comprising, in a pharmaceutically acceptable carrier, a solution of a potent rubefacient or a vasodilator compound, preferably capsaicin, methyl nicotinate or a known vasodilator in concentrations suitable to produce and maintain enhanced blood flow to scalp area.

3. A composition for stimulating the growth of human and animal hair by enhancing the growth of stem cells or bulb cells and enhancing blood flow to scalp, in a pharmaceutical acceptable carrier, such as an alcoholic solution of a β-sitosterol with a known vasodilator or rubefacient compound.

4. It is claimed as in claims 1 and 3 wherein the composition contains a phytosetrol, especially β-sitosterol.

5. It is claimed as in claims 2 and 3 wherein the blood flow enhancer compound may be selected from the following category but not limited to capsicum extract; erucic acid; nicotinic acid salts; nicotinic acid esters; and nicotinyl alcohols; mustard oil; menthol; methyl salicylate and other compound which are known to cause enhancement of blood flow either by acting as rubefacient or by other pharmacological mechanisms such as vasodilatation or other localized or central pharmacological mechanisms to enhance blood flow to tissue. The vasodilators in this class include debrisoquine. Further classes of vasodilators act on pharmacological receptors on the smooth muscle membrane. These include pre-synaptic receptor blockers and vasodilators, which reduce the amount of chemical messenger in the synaptic vesicles, which provide the point of contact with the smooth muscle. An example of the former type is clonidine and an example of the latter type is guanethidine. One specific class of vasodilators acts on catecholamine transmitters and is termed alpha-adrenergic blocking agents. Examples of this type of vasodilator include prazosin, lebetaiol, doxazocin, phenoxybenzamine, phentolamine, betahistine, ergotamine and sumatriptin. There are several other receptor types present on the smooth muscle cell which mediate contractions and vasodilation results when actuation of these receptors is interferred with renin receptors and angiotensin II receptors mediate such contractions, and agents which block these processes indirectly or directly are Vasodilators. ACE inhibitors and Angiotensin II receptor antagonists include include ibesartan. The ACE Inhibitors include quinapril, captopril, enalapril, perindopril, trandolapril, cilazapril, fosinopril, lisinopril, and ramipril. There are other nerve processes which mediate contraction-these are the purinergic and neuropeptide Y transmitter and receptor systems and vasodilators which act on these nerve processes may be used in accordance with the invention. Similarly there is a range of receptor types, which may be targeted to provide the vasodilator effect. These include α-adrenergic, α-2-adrenergic, neuropeptide Y and purinergic. A further major class of vasodilators is those, which act directly in the smooth muscle membrane. They include hydrallazine, verapamil, diltiazem, felodipine, minoxidil, amlodipine, glyceryl trinitrate, isosorbide mononitrate, nicorandil, dipyridamole, multiple actives, alprostadil, oxpentifylline, hydroxyethyl rutosides and tartrazine, adenosine and nimodipine. The quantity of these ingredients used is sufficient to produce a visible enhancement or reddening of scalp surface when applied locally to scalp in a suitable pharmaceutical carrier.

6. It is claimed as in claims 1, 2 and 3 wherein the carrier for the primary agent or agents may optionally include a substantially water-insoluble transdermal penetration enhancing compound selected from the group consisting of C4 to C16 aliphatic group substituted acetals, hemi-acetals and morpholines and further comprising a physiologically acceptable water soluble polar compound selected from the group consisting of alcohols, glycols, lactams, urea, cycloethylene urea, 1,3-dioxolone, 2-methyl-1-3-dioxolone, 1,3-dioxane, 2methyl-1,3-dioxane, morpholine, N-methylmorpholine, N-dimethylformamide, dimethylsulfoxide, methylacetate, ethyllactate, monosaccharides, polysaccharides, amino acids, amino alcohols, diethylamine and cycloethylene carbonate. The polar compound may be selected from a group consisting of alcohol, glycol, dioxolane, formamide, carbonate, glucose, urea and mixtures thereof. Alternatively, the polar compound may be an alcohol glycol mixture or lactim. Other compounds include 1-dodecylazacycloheptan-2-one hexamethylenelauramide, N-methyl-2-pyrrolidone, a sucrose aliphatic acid ester, and nonionic surfactants, in an amount of 0.5-10% by weight of the preparation.

7. It is claimed as in claims 1, 2 and 3 wherein the composition is a pharmaceutically acceptable dosage form suitable for topical administration. The term “pharmaceutically acceptable dosage form,” includes but is not limited to physically and chemically stable solutions, creams, shampoos, lotions, jellies, adhesive type devices, liposomal carrier devices or dispersions, suspensions, emulsions, poultices, or any other suitable form that can be applied locally to scalp. In its preferred embodiment, the composition is used as an alcoholic or hydro-alcoholic solution.

8. It is claimed as in claims 1, 2 or 3 wherein the composition is optionally combined with ingredients that act as preservatives or stabilizers of the composition.

9. It is claimed as in claims 1, 2 or 3 wherein the composition is combined with other components such as nutrients generally considered necessary for the scalp treatment including but not limited to vitamin A, series of vitamin Bs, vitamin C, cyanocobalamin, vitamin E, methionine, cystine or other amino acids, albumin, lactalbumin, selenium or other trace metals, thymus, melatonin, and yeast.

10. It is claimed as in claims 1, 2 or 3 wherein the composition is combined with other drugs or food supplements that work synergistically to promote conversion or growth of stem cells, enhance blood flow and stimulate hair follicular growth.

11. It is claimed as in claims 1 and 3 wherein the β-sitosterol used is in either a purified form, chemically synthesized form or obtained by including in the composition oils or other natural products that contain β-sitosterol.

12. It is claimed as in claims 1, 2 or 3 wherein the composition contains alcohol as a solvent; this can be any type of alcohol, not necessarily ethanol, which is suitable for application to skin which may include but not limited to SD alcohol, benzyl alcohol, methyl alcohol, isopropyl alcohol, etc.
